(19) United States
(12) Patent Application Publication
(10) Pub. No.: US 2012/0003325 A1
(43) Pub. Date: Jan. 5, 2012

(54) USE OF HELIUM-OXYGEN GAS MIXTURES FOR TREATING PULMONARY ARTERIAL HYPERTENSION

(75) Inventors: Hubert Trübel, Wuppertal (DE); Eva-Maria Becker, Wuppertal (DE); Stefan Schäfer, Goch (DE); Katja Schaefer, Radevormwald (DE); Juergen Kohlmeyer, Herne (DE)

(73) Assignee: BAYER SCHERING PHARMA AKTIENGESELLSCHAFT, Berlin (DE)

(21) Appl. No.: 13/126,639

(22) PCT Filed: Oct. 20, 2009
(86) PCT No.: PCT/EP2009/007488

§ 371 Date: Jul. 12, 2011

(30) Foreign Application Priority Data
Oct. 31, 2008 (DE) 10 2008 054 205.9

(51) Int. Cl. A61K 33/00; A61P 11/00

(52) U.S. Cl. 424/613

(57) ABSTRACT

The present application relates to the use of helium-oxygen gas mixtures for the treatment and/or prophylaxis of primary and secondary forms of pulmonary hypertension (PH) and also to the combination of drugs and helium-oxygen gas mixtures, wherein the gas mixtures are used as carrier gases to improve the introduction of a drug for the treatment and/or prophylaxis of pulmonary hypertension.

The present application relates to the use of helium-oxygen gas mixtures for the treatment and/or prophylaxis of primary and secondary forms of pulmonary hypertension (PH) and also to the combination of drugs and helium-oxygen gas mixtures, wherein the gas mixtures are used as carrier gases to improve the introduction of a drug for the treatment and/or prophylaxis of pulmonary hypertension.

Therapy for Primary and Secondary PH

Primary pulmonary arterial hypertension (PAH) is a progressive lung disease which, untreated, leads to death on average within 2.8 years after diagnosis. An increasing constriction of the pulmonary circulation causes additional strain on the right heart, which may ultimately result in failure of the right heart. Chronic pulmonary hypertension is defined by a mean pulmonary artery pressure (mPAP) of >25 mmHg at rest or >30 mmHg under stress (normal value: <20 mmHg). The pathophysiology of pulmonary arterial hypertension is characterized by vasoconstriction and remodeling of the pulmonary vessels. In the case of chronic PAH, the vessel musculature increases in circumference, and this is followed by a slow conversion of the musculature to connective tissue. This increasing obliteration of the pulmonary circulation results in a progressive strain on the right heart, which leads to a reduced output of the right heart and ultimately ends in failure of the right heart. With a prevalence of 1-2 per million, PAH is an extremely rare disease (G. E. D'Alonzo et al., Ann. Intern. Med. 1991, 115, 343-349). The average age of patients has been estimated at 36 years; only 10% of patients were over 60 years old. Distinctly more women than men are affected.

Secondary PH occurs, inter alia, as the result of a lung disease. This can occur acutely in the context of an “adult respiratory distress syndrome” (Kollef et al., N Engl J Med. 1995 Jan. 5; 332(1): 27-37) as a characteristic feature, distinctly worsen the prognosis of the ARDS, and require specific forms of therapy in order to prevent failure of the right heart (Moloney et al., Eur Respir J. 2003 April; 21(4): 720-7). Similarly, chronic lung diseases can also be complicated secondarily by the occurrence of PH and the prognosis can be worsened as a result (e.g., “chronic obstructive pulmonary disease” (COPD); Han et al., Circulation. 2007 Dec. 18; 116(25): 2992-3005). PH underlying a lung disease has been classified under group III in the WHO PAH classification system. In the most general sense, the term “pulmonary hypertension” comprises certain forms of pulmonary hypertension, as have been defined by the World Health Organization (WHO) for example (Clinical Classification of Pulmonary Hypertension, Venice 2003; Simmenau et al., J Am Coll Cardiol (2004), 43, Suppl 1(12) S5-S12).

The standard therapies used in the therapy for acute PH (e.g., prostacyclin analogs, endothelin receptor antagonists, phosphodiesterase inhibitors) are capable of improving the quality of life, the physical capacity, and the prognosis of patients. However, the applicability of these medicaments is restricted by the sometimes serious secondary effects and/or complex forms of administration. The period over which the clinical situation of patients can be improved or stabilized by means of a specific monotherapy is limited. What eventually follows is an escalation in therapy and therefore a combination of therapies in which multiple medicaments have to be given at the same time. New combinations of therapies are one of the most promising future therapy options for treating pulmonary arterial hypertension (Ghofrani et al., Herz 2005, 30, 296-302). In this regard, research into new pharmacological mechanisms of treating PH is of particular interest. New therapies ought to be combinable with the known therapies.

A further secondary effect of a resistance-lowering therapy in the case of secondary PH, which can occur especially in the case of a systemic therapy for a secondary PH with inhomogeneous lung damage (e.g., ARDS and COPD), is a reduction in the arterial oxygen content, despite successful therapy for the pulmonary hypertension, owing to initiation of pulmonary shunts (Stolz et al., Eur Respir J. 2008 September; 32(3): 619-28.).

In view of the secondary effects specified above with the forms of therapy known to date for primary and secondary PH, the object of the present invention is to discover new methods for treating primary and secondary PH which do not have the disadvantages presented above.

Helium-Oxygen Mixtures in the Therapy for PH

Normal ambient air is composed primarily of the elements nitrogen (about 78% by volume) and oxygen (about 21% by volume). Replacement of the nitrogen portion by the noble gas helium results in heliox—a mix of helium and oxygen.

Compared with nitrogen and oxygen, helium has some fundamental properties which are different. Characteristics of the noble gas helium (He) are colorlessness, odorlessness, and tastelessness, and also a low solubility in aqueous solutions and fatty substances (e.g., only 30% of the solubility of oxygen or nitrogen in an oil-water mixture (Brubakk A O, Neumann T S. Bennett & Elliot's Physiology and Medicine of Diving. 5th edition, Saunders (publisher), Edinburgh 2003)). Therefore, a hyperbaric exposure to helium does not result in narcotic effects, as are known for nitrogen or xenon for example. These favorable properties are also present in the mixture of helium and oxygen (heliox) and thus enable diving below 60 m. In commercial diving, the nitrogen present in the air breathed is completely or partly replaced by helium. This can, inter alia, also reduce the formation of gas bubbles during resurfacing (decompression sickness or caisson disease).

Owing to its saturated electron shell, helium barely reacts with other substances. Therefore, it is used in pulmonology in the foreign gas dilution method for determining lung volume.

As early as before the Second World War, A. Barach investigated the medical use of the gas mixture heliox and explored its application in upper and lower airway obstructions (Barach, Proc Soc Exp Biol Med 1934; 32: 462-464; Barach, Ann Intern Med 1935; 9: 739-765). Later on, heliox as an airway therapeutic agent became less important, since priority was given to using helium in military technology during the war and newer therapeutic options, such as inhalative β₂ mimetics, were developed after the Second World War.

Since the eighties, an increasing interest in the use of the gas mixture heliox in severe upper and lower airway obstructions has again been observed.

The heliox effect in the respiratory tract depends, inter alia, on the location of an obstruction. Although the width of the airways becomes narrower toward the periphery, the growing number of bronchioles in the deeper generations results in a greater total cross section and thus a lower total resistance. Accordingly, the substantial portion of the airway resistance is located in the upper airways up to the 5th to 6th bronchial generation (West J B. Respiratory Physiology—the essentials. 5th edition, 1995, Williams and Wilkins, Baltimore). In numerous pathological states of the lung (e.g., ARDS, COPD), the small airways also exhibit in some cases considerable narrowing which can lead to a change in the flow profile. The transition from laminar to turbulent flow can be estimated using the Reynolds number (RE). RE is calculated according to:

RE=(4*ρ*V′)/π*μ*D

(ρ: density; V′: volume flow; μ: viscosity; D: diameter of the tube)

At a critical Reynolds number of ˜2000, a laminar flow increasingly turns into a transitional flow and eventually (Re>4000) into a turbulent flow, and therefore inner friction and shearing forces increasingly occur and higher pressure gradients (compared with the laminar flow) are necessary for movement of the gas flow (West J B. Respiratory Physiology—the essentials. 5th edition, 1995, Williams and Wilkins, Baltimore). Since the density of helium is only about 13% of the density of nitrogen, admixing helium with a gas mixture lowers the Reynolds number. This favors a transition from turbulent flow to laminar flow. When this transition results in a laminar flow profile, the effort of breathing is reduced, since laminar gas flows have less inner friction compared with turbulent flows and therefore require fewer driving forces (i.e., also less effort of breathing) (Jolliet et al., Respir Care Clin N Am. 2002; 8: 295-307) in order to be moved in the airways. In summary, there are two mechanisms which facilitate gas flow in the case of mixing with helium: firstly, a laminar flow becomes more likely, and secondly, a flow which is turbulent throughout is moved with less pressure. Both effects reduce the effort of breathing as the work which has to be put in for gas exchange.

In the same way as for diseases of the upper airways (e.g., tightness in the region of the vocal cords), a helium-oxygen mixture can be used with helium-oxygen gas mixtures for diseases of the lower airways (e.g., COPD or asthma). Usually, the breathing-facilitating action owing to the described effect of a prominent transformation of a turbulent flow into a laminar gas flow is also important here. In addition, the mechanism of action of a more effective deposition of aerosol particles (e.g., β₂ mimetics) into more peripherally situated portions of the lung is also discussed (Anderson et al., Am Rev Respir Dis. 1993; 147: 524-8.).

In the context of investigations which had the goal of improving deposition of inhalable active ingredients in primary and secondary PH using helium-oxygen mixtures, a surprising and unexpected result was that the use of helium-oxygen mixtures alone without any further additional active ingredient for PH therapy (e.g., prostacyclin analogs, sGC activators and stimulators) leads to a clear reduction in pulmonary vascular resistance. Furthermore, this effect increases when additionally inhalable active ingredients are combined with helium-oxygen mixtures.

The experimental result according to the invention that helium-oxygen mixtures can lower the resistance on the vascular side can be used for the treatment and/or prophylaxis of pulmonary hypertension, for example in acute (e.g., ARDS) pulmonary or cardiac (left-atrial or left-ventricular) diseases and also in heart valve diseases. Furthermore, helium-oxygen mixtures are thus suitable not only for the treatment of an airway obstruction but also for the treatment and/or prophylaxis of pulmonary hypertension in chronic obstructive pulmonary disease, interstitial lung disease, sleep apnea syndrome, diseases with alveolar hypoventilation, altitude sickness, and pulmonary developmental disorders.

In addition, the helium-oxygen mixtures are suitable for the treatment and/or prophylaxis of pulmonary arterial hypertension caused by chronic thrombotic and/or embolic diseases, such as thromboembolism of the proximal pulmonary arteries, obstruction of the distal pulmonary arteries, and pulmonary embolism for example. The compounds according to the invention can also be used for the treatment and/or prophylaxis of pulmonary arterial hypertension associated with sarcoidosis, histiocytosis X, or lymphangioleiomyomatosis and also pulmonary arterial hypertension caused by external vascular compression (lymph nodes, tumor, fibrosing mediastinitis).

Helium-oxygen mixtures can be used alone or in combination with other active ingredients. Helium-oxygen mixtures can be used with a ratio from 20 to 80% helium. Preference is given to using a ratio with very high proportion of helium (up to 79%). Particular preference is given to using a ratio of 79% helium/21% oxygen.

Percentage values in the present invention mean percent by volume throughout.

The present invention further relates to drugs comprising a helium-oxygen mixture and one or more further active ingredients for the treatment and/or prophylaxis of the above-mentioned diseases. Suitable combinations of active ingredients which may be mentioned by way of example and by preference are:

Kinase inhibitors, more particularly tyrosine kinase inhibitors, such as, for example and preferably, sorafenib, imatinib, gefitinib, or erlotinib, in combination with helium-oxygen mixtures

Nitric oxide (NO) in combination with helium-oxygen mixtures

NO-independent, but heme-dependent, stimulators of soluble guanylate cyclase, such as more particularly the compounds described in WO 00/06568, WO 00/06569, WO 02/42301, and WO 03/095451, in combination with helium-oxygen mixtures.

The following compounds may be listed here by preference:

### Methyl 4,6-diamino-2-[1-(2-fluorobenzyl)-1H-pyrazolo[3,4-b]pyridin-3-yl]-5-pyrimidinyl carbamate

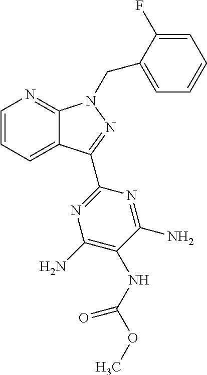

### Methyl 4,6-diamino-2-[1-(2-fluorobenzyl)-1H-pyrazolo[3,4-b]pyridin-3-yl]-5-pyrimidinyl (methyl)carbamate

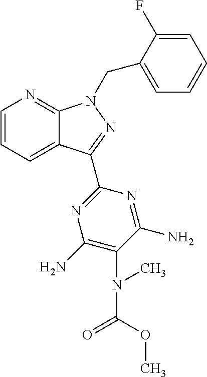

NO- and heme-independent activators of soluble guanylate cyclase, such as more particularly the compounds described in WO 01/19355, WO 01/19776, WO 01/19778, WO 01/19780, WO 02/070462, and WO 02/070510, in combination with helium-oxygen mixtures

### 4-[((4-Carboxybutyl)-{2-[(4-phenethylbenzyl)oxy]phenethyl}amino)methyl]benzoic acid

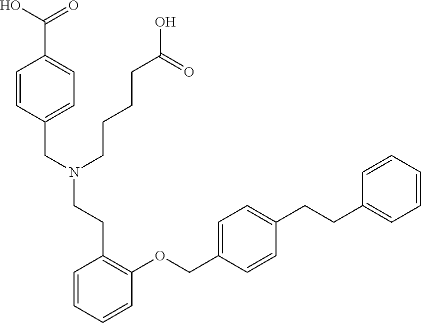

Prostacyclin analogs, such as, for example and preferably, iloprost, beraprost, treprostinil, or epoprostenol, in combination with helium-oxygen mixtures

Endothelin receptor antagonists, such as, for example and preferably, bosentan, darusentan, ambrisentan, or sitaxsentan, in combination with helium-oxygen mixtures

Compounds which inhibit the degradation of cyclic guanosine monophosphate (cGMP) and/or cyclic adenosine monophosphate (cAMP), such as inhibitors of phosphodiesterases (PDE) 1, 2, 3, 4, and/or 5 for example, more particularly PDE 5 inhibitors, such as sildenafil, vardenafil, and tadalafil, in combination with helium-oxygen mixtures Antibiotics, such as glycoside antibiotics, gyrase inhibitors, or penicillins for example, in combination with helium-oxygen mixtures

Antiviral substances, such as aspirin for example, in combination with helium-oxygen mixtures

Antiproliferative substances in the treatment of tumors in combination with helium-oxygen mixtures

General active ingredients which can develop an extrapulmonary (systemic) effect in the manner mentioned above in combination with helium-oxygen mixtures.

For the inhalation of active ingredients by means of heliox, preference is given to using helium/oxygen mixing ratios having a very high proportion of helium (up to 79%). Particular preference is given to using a ratio of 79% helium/21% oxygen, with this proportion the proportion of helium possibly having to be reduced in case of an increased demand for oxygen by a patient.

The present invention further relates to the use of helium-oxygen mixtures alone or in combination with one or more of the abovementioned combinations of active ingredients for producing a drug for the treatment and/or prophylaxis of pulmonary hypertension in left-atrial or left-ventricular diseases, left-sided heart valve diseases, acute lung diseases (e.g., ARDS), chronic obstructive pulmonary disease, interstitial lung disease, sleep apnea syndrome, diseases with alveolar hypoventilation, altitude sickness, pulmonary developmental disorders, chronic thrombotic and/or embolic diseases such as thromboembolism of the proximal pulmonary arteries, obstruction of the distal pulmonary arteries, and lung embolism for example, associated with sarcoidosis, histiocytosis X, or lymphangioleiomyomatosis, and also pulmonary arterial hypertension caused by external vascular compression (lymph nodes, tumor, fibrosing mediastinitis).

The present invention further relates to a process for the treatment and/or prophylaxis of pulmonary arterial hypertension in humans and animals by administering helium-oxygen mixtures or a combination of helium-oxygen mixtures and one or more of the abovementioned combinations of active ingredients.

The drugs to be produced according to the use according to the invention or to be used according to the invention comprise at least one of the compounds according to the invention, usually together with one or more inert, nontoxic, pharmaceutically suitable excipients, in combination with helium-oxygen mixtures.

The present invention further relates to drugs comprising at least one of the compounds according to the invention in combination with one or more inert, nontoxic, pharmaceutically suitable excipients, for the treatment and/or prophylaxis of the abovementioned diseases in combination with helium-oxygen mixtures.

The use of heliox in the inhalation of a liquid, solid, or gaseous active ingredient (inhalant) may not only affect pulmonary vascular resistance independently of the active ingredient but also intensify the action of the inhaled liquid, solid, or gaseous active ingredient. This intensification is achieved by, for example, a higher deposition rate, deposition distally from flow impediments or in poorly ventilated areas. Heliox can be used for producing the inhalant, and it is also possible for an inhalant to be produced with or without heliox but to be administered with heliox into the lung.

The combination of heliox and a liquid, solid, or gaseous active ingredient can be produced with the aid of commercially available devices (for example, 2.4 MHz, Optineb-IR, from Nebu-Tec).

Ventilation with heliox or with the combination of heliox and an active ingredient can be achieved with the aid of commercially available ventilation devices (for example, Avea, from Viasys Healthcare).

Parenteral administration can using or in combination with helium-oxygen mixtures the route of administration is suitable via the airways, for example inhalation dosage forms (inter alia, powder inhalers, nebulizers), nasal drops, nasal solutions, or nasal sprays.

Helium-oxygen mixtures are commercially available in a mixing ratio usually of 79% helium and 21% oxygen. However, the term heliox does not specifically describe this mixing ratio, but merely the mixture of helium and oxygen. Each of these helium/oxygen mixtures, where a minimum proportion of oxygen of 21% is necessary for physiological reasons, can be converted into the administration forms specified. This can be achieved in a manner known per se by mixing with inert, nontoxic, pharmaceutically suitable excipients. These excipients include, inter alia, carriers (e.g., microcrystalline cellulose, lactose, mannitol), solvents (e.g., liquid polyethylene glycols), emulsifiers and dispersants or surfactants (e.g., sodium dodecyl sulfate, polyoxysorbitan oleate), binders (e.g., polyvinylpyrrolidone), synthetic and natural polymers (e.g., albumin), stabilizers (e.g., antioxidants, such as ascorbic acid for example), dyes (e.g., inorganic pigments, such as iron oxides for example), and flavor and/or odor masking agents.

In general, it has been found to be advantageous in inhalative therapy to keep the proportion of helium in a combination of helium and oxygen as large as possible, with experimental results indicating that the proportion of helium should be between 79% and 25%.

Nevertheless, it may be necessary, where appropriate, to deviate from the mixing ratios mentioned of helium and oxygen, dependent on body weight, route of administration, individual reaction toward the active ingredient, type of preparation, and time or interval at which the administration is carried out. Thus, it may be sufficient in some cases to use less than 25% helium.

### EXPERIMENTAL SECTION

The following exemplary embodiments elucidate the experimental design, which, in a model of inhomogeneous, acute lung damage, to the surprising and unexpected result that the use of helium-oxygen mixtures alone without any further additional active ingredient for PH therapy (e.g., prostacyclin analogs, sGC activators and stimulators) leads to a clear reduction in pulmonary vascular resistance. The invention is not restricted to the examples, since it became further apparent that this resistance-lowering effect of helium-oxygen mixtures can be intensified in the pulmonary vascular bed by the additional inhalation of active ingredients:

To induce severe lung damage which is of considerable clinical relevance to newborn children as well, an ALI/ARDS is used by removing pulmonary surfactant by means of lavage on a narcotized piglet with subsequent intratracheal administration of a 20% meconium solution. The animals experience a pronounced gas exchange disorder with secondary pulmonary hypertension. The model described corresponds to meconium aspiration syndrome. To detect drug effects, measurements are made of various physiological parameters (heart rate, blood pressure in the aorta and the pulmonary artery, pressure profile in the left ventricle, cardiac output, blood gas analysis in arterial and venous blood) according to a standardized procedure (Geiger et al., Intensive Care Med. 2008; 34: 368-76) in the Göttingen Minipig® (Ellegaard, DK) under adequate analgosedation.

1. (canceled)

2. (canceled)

3. (canceled)

4. (canceled)

5. (canceled)

6. (canceled)

7. A composition comprising a helium-oxygen gas mixture and one or more drugs selected from the group consisting of
kinase inhibitors, tyrosine kinase inhibitors, sorafenib, imatinib, gefitinib, or erlotinib;
nitric oxide (NO);
NO-independent, but heme-dependent, stimulators of soluble guanylate cyclase;
NO- and heme-independent activators of soluble guanylate cyclase;
prostacyclin analogs, iloprost, beraprost, treprostinil, or epoprostenol;
endothelin receptor antagonists, bosentan, darusentan, ambrisentan, or sitaxsentan;
compounds which inhibit the degradation of cyclic guanosine monophosphate (cGMP) and/or cyclic adenosine monophosphate (cAMP), inhibitors of phosphodiesterases (PDE) 1, 2, 3, 4, and/or 5, sildenafil, vardenafil, or tadalafil;
antibiotics, glycoside antibiotics, gyrase inhibitors, or penicillins;
antiviral substances, aspirin;
antiproliferative substances in the treatment of tumors;
and
general active ingredients which can develop an extrapulmonary (systemic) effect in the manner mentioned above.

8. A composition comprising a helium-oxygen gas mixture and one or more drugs selected from the group consisting of
methyl 4,6-diamino-2-[1-(2-fluorobenzyl)-1H-pyrazolo[3,4-b]pyridin-3-yl]-5-pyrimidinyl carbamate 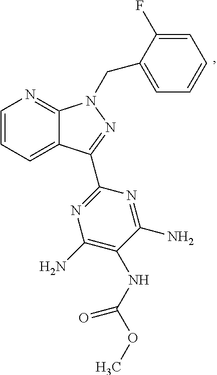
methyl 4,6-diamino-2-[1-(2-fluorobenzyl)-1H-pyrazolo[3,4-b]pyridin-3-yl]-5-pyrimidinyl (methyl)carbamate 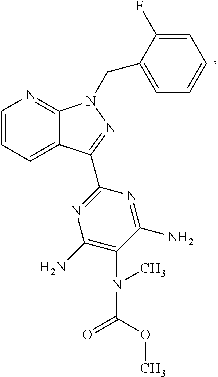
and
4-[((4-carboxybutyl)-{2-[(4-phenethylbenzyl)oxy]phenethyl}amino)methyl]benzoic acid

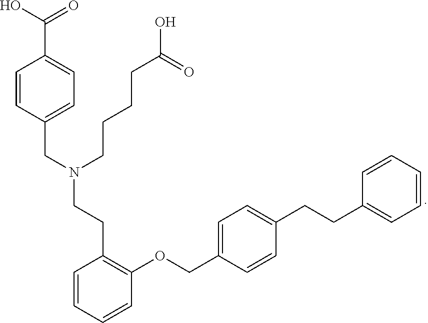

9. (canceled)

10. (canceled)

11. (canceled)

12. (canceled)

13. The composition of claim 7, wherein the helium-oxygen gas mixture has a composition of from 20 to 79% helium and from 80 to 21% oxygen.

14. The composition of claim 7, further comprising an inert, nontoxic, pharmaceutically suitable excipient.

15. The composition of claim 8, wherein the helium-oxygen gas mixture has a composition of from 20 to 79% helium and from 80 to 21% oxygen.

16. The composition of claim 8, further comprising an inert, nontoxic, pharmaceutically suitable excipient.

17. A method for the treatment and/or prophylaxis of primary or secondary forms of pulmonary hypertension (PH) comprising administering to a patient in need thereof a helium-oxygen gas mixture.

18. The method of claim 17, wherein the helium-oxygen gas mixture has a composition of from 20 to 79% helium and from 80 to 21% oxygen.

19. A method for the treatment and/or prophylaxis of primary or secondary forms of pulmonary hypertension (PH) comprising administering to a patient in need thereof a helium-oxygen gas mixture as claimed in claim 7.

20. The method of claim 19, wherein the helium-oxygen gas mixture has a composition of from 20 to 79% helium and from 80 to 21% oxygen.

21. A method for the treatment and/or prophylaxis of primary or secondary forms of pulmonary hypertension (PH) comprising administering to a patient in need thereof a helium-oxygen gas mixture as claimed in claim 8.

22. The method of claim 21, wherein the helium-oxygen gas mixture has a composition of from 20 to 79% helium and from 80 to 21% oxygen.
